(19) United States
(12) Patent Application Publication
(10) Pub. No.: US 2024/0288437 A1
(43) Pub. Date: Aug. 29, 2024

(54) METHODS OF DETERMINING RISK OF AND TREATING CANCER RECURRENCE

(71) Applicant: THE REGENTS OF THE UNIVERSITY OF MICHIGAN, Ann Arbor, MI (US)

(72) Inventor: Howard R. PETTY, Ann Arbor, MI (US)

(21) Appl. No.: 18/575,591

(22) PCT Filed: Jun. 28, 2022
(86) PCT No.: PCT/US2022/035342

§ 371 Date: Dec. 29, 2023

Related U.S. Application Data
(60) Provisional application No. 63/216,362, filed on Jun. 29, 2021.

(51) Int. Cl. G01N 33/574; A61K 45/06; G01N 1/30

(52) U.S. Cl. CPC G01N 33/57496; A61K 45/06; G01N 1/30; G01N 33/57415; G01N 2333/912; G01N 2800/54

(57) ABSTRACT

The present disclosure provides systems and methods for the prediction and treatment of recurrent cancer in a subject. Provided herein is the analysis of subcellular localization of phosphofructokinase type L (PFKL), phosphofructokinase/fructose-2,6-bisphosphatase type 4 (PFKFB4), and other biomarkers, and correlation thereof to the likelihood of cancer (e.g., DCIS) recurrence.

### CROSS-REFERENCE TO RELATED APPLICATIONS

This application claims the benefit of U.S. Provisional Patent Application No. 63/216,362, filed on Jun. 29, 2021, which is incorporated by reference herein.

### FIELD

The present disclosure provides systems and methods for the prediction and treatment of recurrent cancer in a subject. Provided herein is the analysis of subcellular localization of phosphofructokinase type L (PFKL), phosphofructokinase/fructose-2,6-bisphosphatase type 4 (PFKFB4), and other biomarkers, and correlation thereof to the likelihood of cancer (e.g., DCIS) recurrence.

### BACKGROUND

Ductal carcinoma in situ (DCIS) of the breast is a common non-invasive cancer. Epidemiology studies suggest that indolent and aggressive forms of DCIS exist, with the aggressive form potentially leading to life-threatening disease. The two presumed forms of DCIS would exhibit cellular proliferation (indolent) or cellular proliferation plus biochemical and biophysical changes to support invasive behavior (aggressive). Some patients treated for ductal carcinoma in situ (DCIS) of the breast will experience cancer recurrences, whereas other patients will not. Unfortunately, current techniques cannot identify which pre-invasive lesions will lead to recurrent cancer. Although screening tools can detect cancer, they cannot predict cancer recurrences.

### SUMMARY

The present disclosure provides systems and methods for the prediction and treatment of recurrent cancer in a subject. Provided herein is the analysis of subcellular localization of phosphofructokinase type L (PFKL), phosphofructokinase/fructose-2,6-bisphosphatase type 4 (PFKFB4), and other biomarkers, and correlation thereof to the likelihood of cancer (e.g., DCIS) recurrence.

In some embodiments, the methods comprise determining intracellular localization of at least one biomarker (e.g., PFKL, PKKFB4, pGLUT1, etc.) for cancer recurrence in a sample comprising cancer cells from a subject and predicting cancer recurrence in a subject. In some embodiments, the peripheral intracellular localization of at least one biomarker predicts cancer recurrence. In some embodiments, the methods further comprise a) immunostaining the sample with a primary antibody directed to the biomarker for cancer recurrence and b) imaging the sample. In some embodiments, the primary antibody is detected with a secondary antibody comprising a detectable label. In some embodiments, imaging the sample comprises fluorescence microscopy.

In some embodiments, the biomarker for cancer recurrence is phosphofructokinase type L (PFKL) or phosphofructokinase/fructose-2,6-bisphosphatase type 4 (PFKFB4). In some embodiments, intracellular localization of both PFKL and PKKFB4 is analyzed/monitored. In some embodiments, intracellular localization of one or more other biomarkers is analyzed/monitored along with PFKL and/or PKKFB4, such as glutamate cysteine ligase catalytic domain (GCLC), glutathione synthetase (GS), cystine-glutamate antiporter (xCT), CD44v9, glutamine uptake transporters ASCT2, ATBO+ and LAT1, leucine uptake (LAT1), g-glutamyl transpeptidase (GGT), g-glutamyl cysteine (gGC), glucose transporter 1 (GLUT1), glucose 6-phosphate dehydrogenase (G6PD), transketolase (TKT), transketolase-like protein 1 (TKTLP1), RhoA, RhoA with bound GTP and CD74.

The cancer may be breast cancer, prostate cancer, lung cancer, melanoma, kidney cancer, thyroid cancer, pancreatic cancer, stomach cancer or bladder cancer. In some embodiments, the breast cancer comprises ductal carcinoma in situ of the breast, lobular carcinoma in situ, atypical ductal hyperplasia, or atypical lobular hyperplasia. In select embodiments, the cancer recurrence is ipsilateral breast cancer recurrence. In select embodiments, the sample comprises a formalin-fixed paraffin-embedded cancer tissue sample or a cancer metastases tissue or cell sample.

In some embodiments, the methods further comprise treating a subject predicted to have cancer recurrence. The treatment may include surgery or administration of inhibitors to enzyme or transporter accumulation at plasma membrane. In some embodiments, the inhibitors to enzyme accumulation at plasma membrane comprise colchicine, taxol, a calmodulin antagonist, a prenylation inhibitor, an anesthetic, or combinations thereof.

Provided herein are methods of preventing cancer recurrence in a subject comprising predicting cancer recurrence by the methods disclosed herein and administering a treatment regimen. The treatment regimen may comprise one or more of surgery; administration of an inhibitor(s) to enzyme or transporter accumulation at plasma membrane; immunotherapy; radiotherapy; and administration of a chemotherapeutic agent(s).

Further disclosed herein a systems for use in predicting cancer recurrence or distinguishing between recurrent and non-recurrent cancer. The systems comprise at least one or all of a primary antibody to a biomarker for cancer recurrence; an imaging instrument; software configured to determine the intracellular location of the biomarker for cancer recurrence and a sample.

### BRIEF DESCRIPTION OF THE DRAWINGS

FIG. 1. Chemical relationships among cellular elements contributing to glycolysis. Panel A shows chemical transformations in glycolysis. Intracellular glucose is converted into G6P, which can enter the PPP or glycolytic pathway. PGI converts G6P into F6P. This product is converted by phosphofructokinase type I (the diagram shows PFKL) into F(1, 6)BP, which is further metabolized into lactate. PFK type I is a key regulatory enzyme of glycolysis. It is activated by AMP and F(2, 6)BP and inhibited by ATP, PEP, citrate, and glycosylation. The reverse reaction, F(1, 6)BP→F6P, is catalyzed by FBP. F(2, 6)BP, the strongest activator of PFK type I, is produced by PFKFB4 (a PFK type II enzyme). Panel B shows a hypothetical illustration of a glycosome (a glycolytic metabolon) and several functionally associated cellular elements. A glycosome is shown at the top of this panel in association with the plasma membrane. The proteins in black are included in this study (PFKL, PFKFB4) or a previous study (phospho-GLUT1) (Ref. 31; incorporated by reference in its entirety), and found to be metabolic switches participating in breast cancer recurrences. PFKL has been associated with a variety of cellular components including: the plasma membrane, F-actin, linear polymers, monomers, clumps, and the nucleolus.

FIG. 2. Representative intracellular patterns of PFKL and PFKFB4 within tissue sections from surgical biopsies of DCIS patients subsequently found to exhibit non-recurrent or recurrent cancer. DCIS tissue samples from patients who did not experience a cancer recurrence (A, B) and did experience a cancer recurrence (C, D) are shown. Both PFKL (A) and PFKFB4 (B) were found in a central distribution within epithelial cells of patients who did not experience a recurrence. Panels C and D show PFKL and PFKFB4 labeling of a sample from a patient who subsequently experienced recurrent cancer. These enzymes often adopt a peripheral pattern in epithelial cells of biopsies of patients who later experienced a recurrence. Other cell types within sections, such as mesenchymal cells, endothelial cells, and blood cells within the microvasculature (examples are labeled V in panels B and D) are also be stained with anti-PFK antibodies. Machine outcome predictions for panels A and B were non-recurrent, whereas machine predictions for panels C and D were recurrent. (Bar=50 μm)

FIG. 3. Flux-controlling glycolytic steps may relocate to the apical surface of ductal epithelial cells in DCIS. This figure shows the apical accumulation of PFKL, PFKFB4, and phospho-GLUT1 biomarkers in a subpopulation of DCIS cases that exhibited recurrences. In this study, two of 50 recurrent patient samples exhibited extensive accumulation of three glycolytic metabolon biomarkers at the apical surface of pre-invasive epithelial cells, approximately one-half of all recurrent lesions has some degree of enrichment at the apical surfaces. Panels A and B show results for PFKL and PFKFB4, respectively. A similar apical accumulation of phospho-GLUT was also noted in another FFPE section from this same patient (panel C). However, this was not a general property of biomarkers nor was it due to redundant membrane, because another membrane biomarker, CD31, was not redistributed to the apical surface (panel D). Nearby capillaries can be identified by their strong labeling with CD31 (PECAM-1). This indicates that in some cases, pre-invasive cells harvest energy from the lumen of the duct. (Bar=50 μm; V=microvasculature) Panel E shows schematic illustrations of conventional and unconventional glucose flow in DCIS lesions. Upper illustration: during conventional glucose flow, carbon from the microvasculature enters interstitial fluids via facilitated diffusion across endothelial surfaces to accumulate in the interstitial tissues. Glucose may be transported across myoepithelial cells, and then into endothelial cells. The glucose could be utilized during transit or equilibrate across the apical membrane to enter the duct's luminal space. Ductal epithelial cells may also derive energy from pyruvate and lactate released from cancer associated fibroblasts (CAF). Lower illustration: a proposed non-canonical pathway of glucose flow in DCIS lesions. This pathway is expected to be especially important during conditions of low glucose concentrations within the interstitial fluid. Pre-invasive epithelial cells may derive energy from the glucose within the lumen, which is internalized via transporters at the apical surface, or that energy is harvested from lactose. Luminal lactose may be internalized via pinocytosis and autophagocytosis at the apical surface. Lactose is hydrolyzed by O-galactosidase within secondary lysosomes, producing galactose and glucose. The glucose is then proposed to enter glycolysis. Additional sources of energy may be derived from citrate, pyruvate, and lactate within milk, which are translocated by organic anion translocators.

FIG. 4. Computer Findings. Panel A shows cross-validation studies of machine-based predictions of breast cancer recurrences. The precision and recall curves of a computer training dataset created using micrographs of DCIS samples from patients exhibiting cancer recurrences and those not exhibiting a recurrence are shown. Precision (solid line) and recall (dashed line) are shown as a function of threshold (see also Table 1). Panel B shows a confusion matrix of clinical outcome predictions using the computer model created by the studies of panel A. Machine-based classifications of DCIS micrographs were obtained for phospho-GLUT1, PFKL, and PFKFB4-labeled DCIS sections. If any one of these computations was found to predict a recurrence, the sample was judged as originating from a patient who will experience a recurrence.

FIG. 5. Minimum number of micrographs required for correct recurrence prediction. The abscissa shows the number of micrographs of recurrent tissue tested to arrive at a correct recurrence prediction. The ordinate shows the fraction of correct outcome predictions. Twenty-nine consecutive samples from patients subsequently reporting a recurrence were studied. The sections were stained with anti-phospho-GLUT1 then imaged. Each image was examined in acquisition order by computer to assess each micrograph's predicted outcome. As more micrographs were recorded for each patient, the number of correct recurrence predictions increased. Based upon these data, at least 10 images per patient were analyzed. (In the case of non-recurrent patients, all images must score as non-recurrent.) The curve does not reach a fraction of 1.0 because 1 patient recurrence was computationally predicted by the PFKL staining patterns, and did not demonstrate a recurrence prediction for the phospho-GLUT1 biomarker. This illustrates the utility of using multiple biomarkers.

FIG. 6. Staining patterns of PFKP and PFKM within tissue sections from surgical biopsies of DCIS patients subsequently found to exhibit non-recurrent or recurrent cancer. DCIS tissue samples from a patient who did not experience a cancer recurrence (A, B) and did experience a cancer recurrence (C, D) are shown. PFKP was found in a peripheral distribution within epithelial cells of patients who did and did not experience a recurrence (A, C). The intracellular distribution of PFKM was primarily at the center of the cell (B, D) for patients who did not exhibit a recurrence and for those that did experience a recurrence. Although the of PFK isotypes distribution varies, only minimal differences were found in comparing recurrent to non-recurrent patient samples. (Bar=50 μm).

FIG. 7. Heterogeneity of PFKL and PFKFB4 labeling patterns of DCIS lesions from non-recurrent patients. This figure shows tissue samples of four DCIS patients who did not experience a cancer recurrence after labeling with anti-PFKL (A, C, E, and G) and anti-PFKFB4 (B, D, F, and H). (Bar=50 μm).

FIG. 8. Visualization of nucleoli within DCIS lesions from non-recurrent (left column) and recurrent (right column) DCIS patients. A, B: Sections from patients subsequently experiencing cancer recurrences or non-recurrences were stained with H&E. Nucleoli are round or oval in shape, which are purple in color. These structures are found near the center of the cell. Superstructure is also evident in nucleoli. C-F: Nucleoli were also identified using anti-nucleolin (C, D) and anti-histone H2A.X (E, F) antibodies. These tests confirm the identification of these structures as nucleoli. There were no apparent differences to identify recurrent vs. non-recurrent DCIS patients in these studies. Thus, nucleolar composition, PFKL and PFKFB4, not nucleoli per se, are an important feature of DCIS recurrence. (Bar=50 μm).

FIG. 9. Heterogeneity of PFKL and PFKFB4 labeling patterns of DCIS lesions from patients exhibiting recurrences. Four DCIS tissue samples from patients who experienced a cancer recurrence were labeled with anti-PFKL (A, C, E, and G) and anti-PFKFB4 (B, D, F, and H) are shown. A variety of ductal morphologies and PFK locations (central and peripheral) are shown. As blood vessels are labeled with these reagents, they can also be visualized (indicated with a V) in panels F and H. (Bar=50 μm).

FIG. 10. PFKL and PFKFB4 labeling of a sample of breast cancer metastasis to the omentum. Tissue sections of breast cancer metastases to the omentum shows solid sheets of cells. Panel A shows PFKL labeling of cancer metastases to the omentum. Panel B shows the same dual-stained slide labeled with anti-PFKFB4. The label is primarily found in a peripheral distribution for both biomarkers. (N=3) (Bar=50 μm).

FIG. 11. Normal adjacent tissue from two breast cancer patients (NAT patient 1 and 2) was stained with anti-phospho-GLUT1 (A, B), anti-PFKL (C, D), and anti-PFKFB4 (E, F) antibodies. The machine-based predicted outcome was non-recurrent for panel A and recurrent for the remaining panels (B-F). Panel A shows phospho-GLUT1 staining of NAT, which resembles that of nonrecurrent DCIS lesions. Panel B shows NAT stained with anti-phospho-GLUT1, which led to a strong recurrence prediction. Although its recurrence status is not obvious, it does possess clumps of unusual size. Panel C and D show anti-PFKL labeled samples of NAT sections. Panels E, F show NAT samples labeled with anti-PFKFB4 antibodies. The ducts of panels C-F show significant peripheral staining, which often accompanies recurrence predictions. The ability of NAT to report metabolic changes in the proximity of aggressive lesions may have clinical relevance. (Bar=50 μm).

### DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing”, and “introducing,” are used interchangeably herein and refer to the placement of therapeutic agents into a subject by a method or route which results in at least partial localization a desired site. The therapeutic agents can be administered by any appropriate route which results in delivery to a desired location in the subject.

“Antibody” and “antibodies,” as used herein, refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2).

As used herein, the term “biomarker” refers to a substance, the detection of which indicates a particular disease/condition or risk of acquiring/having a particular disease/condition. In the context of the method described herein, a “biomarker” can be a protein (e.g. an enzyme or transporter) that changes location with a cell as a predictor of cancer recurrence or an indicator of recurrent cancer.

As used herein, the term “chemotherapeutic” or “anti-cancer drug” includes any drug used in cancer treatment or any radiation sensitizing agent. Chemotherapeutics may include alkylating agents (including, but not limited to, cyclophosphamide, mechlorethamine, chlorambucil, melphalan, dacarbazine, nitrosoureas, and temozolomide), anthracyclines (including, but not limited to, daunorubicin, doxorubicin, epirubicin, idarubicin, mitoxantrone, and valrubicin), cytoskeletal disrupters or taxanes (including, but not limited to, paclitaxel, docetaxel, abraxane, and taxotere), epothilones, histone deacetylase inhibitors (including, but not limited to, vorinostat and romidepsin), topoisomerase inhibitors (including, but not limited to, irinotecan, topotecan, etoposide, teniposide, and tafluposide), kinase inhibitors (including, but not limited to, bortezomib, erlotinib, gefitinib, imatinib, vemurafenib, and vismodegib), nucleotide analogs and precursor analogs (including, but not limited to, azacitidine, azathioprine, capecitabine, cytarabine, doxifluridine, fluorouracil, gemcitabine, hydroxyurea, mercaptopurine, methotrexate, and tioguanine), peptide antibiotics (including, but not limited to, bleomycin and actinomycin), platinum-based agents (including, but not limited to, carboplatin, cisplatin and oxaliplatin), retinoids (including, but not limited to, tretinoin, alitretinoin, and bexarotene), vinca alkaloids and derivatives (including, but not limited to, vinblastine, vincristine, vindesine, and vinorelbine), or combinations thereof. The chemotherapeutic may in any form necessary for efficacious administration and functionality. “Chemotherapy” designates a therapeutic regimen which includes administration of a “chemotherapeutic” or “anti-cancer drug.”

As used herein, the term “preventing” refers to partially or completely delaying or inhibiting onset of an infection, disease, disorder and/or condition; partially or completely delaying or inhibiting onset of one or more symptoms, features, or clinical manifestations of a particular infection, disease, disorder, and/or condition; partially or completely delaying or inhibiting onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition. 100% inhibition or elimination of risk is not necessary to achieve “preventing.” As used herein, f the likelihood of a population developing a condition is achieved, then the condition is prevented for an individual within that population.

The terms “sample,” “biological sample,” and “test sample” are used interchangeably herein to refer to any material, biological fluid, tissue, or cell obtained or otherwise derived from an individual. The term sample also includes materials derived from a tissue culture or a cell culture. This includes blood (including whole blood, leukocytes, peripheral blood mononuclear cells, buffy coat, plasma, and serum), mucosal biopsy tissue and brushed cells, sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the foregoing. For example, a blood sample can be fractionated into serum, plasma, or into fractions containing particular types of blood cells, such as red blood cells or white blood cells (leukocytes). Any suitable methods for obtaining a sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A sample obtained or derived from an individual includes any such sample that has been processed in any suitable manner after being obtained from the individual. It will be appreciated that obtaining a biological sample from a subject may comprise extracting the sample directly from the subject or receiving the sample from a third party. In the context of the method described herein, the sample comprises cells.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treat,” “treating” and the like means a slowing, stopping or reversing of progression of a disease or disorder. The term also means a reversing of the progression of such a disease or disorder. As such, “treating” means an application or administration of the methods or agents described herein to a subject, where the subject has a disease or a symptom of a disease, where the purpose is to cure, heal, alleviate, relieve, alter, remedy, ameliorate, improve or affect the disease or symptoms of the disease.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### DETAILED DESCRIPTION

The present disclosure provides systems and methods for the prediction and treatment of recurrent cancer in a subject. Provided herein is the analysis of subcellular localization of phosphofructokinase type L (PFKL), phosphofructokinase/fructose-2,6-bisphosphatase type 4 (PFKFB4), and other biomarkers, and correlation thereof to the likelihood of cancer (e.g., DCIS) recurrence.

Experiments conducted during development of embodiments herein using ductal carcinoma in situ (DCIS) of the breast as a framework to better understand the mechanism of cancer recurrences using patient outcomes as the physiological observable. Conventional pathology slides were labeled with antiphosphofructokinase type L (PFKL) and anti-phosphofructokinase/fructose-2,6-bisphosphatase type 4 (PFKFB4) reagents. PFKL and PFKFB4 were found in ductal epithelial cell nucleoli from DCIS samples of women who did not experience a cancer recurrence. In contrast, PFKL and PFKFB4 may be found near the plasma membrane in samples from patients who will develop recurrent cancer. Using machine learning to predict patient outcomes, holdout studies of individual patient micrographs for the three biomarkers PFKL, PFKFB4, and phosphorylated GLUT1 demonstrated 38.6% true negatives, 49.5% true positives, 11.9% false positives and 0% false negatives (N=101). A sub-population of recurrent samples demonstrated PFKL, PFKFB4, and phosphorylated glucose transporter 1 accumulation at the apical surface of epithelial cells, suggesting that carbohydrates can be harvested from the ducts' luminal spaces as an energy source. These experiments indicate that PFK isotype patterns are metabolic switches representing key mechanistic steps of recurrences. Furthermore, PFK enzyme patterns within epithelial cells contribute to an accurate diagnostic test to classify DCIS patients as high or low recurrence risk.

The robust diagnostic ability of enzyme and transporter trafficking to the plasma membrane of DCIS samples prior to breast cancer recurrences provides an accurate diagnostic test to identify at risk DCIS patients. In some embodiments, the methods herein prevent the over-diagnosis of life-threatening cancer; thereby reducing the need for unnecessary treatments.

Some embodiments herein employ machine learning to improve outcome predictions. Some embodiments employ diagnostic machine vision applications within imaging software such that outcomes are calculated/evaluated at the time of initial diagnosis.

The present disclosure provides methods of predicting cancer recurrence in a subject, methods of preventing cancer recurrence in a subject and methods for distinguishing recurrent from non-recurrent cancer. The methods comprise determining intracellular localization of at least one biomarker (e.g., PFKL, PKKFB4, pGLUT1, etc.) for cancer recurrence in a sample from a subject comprising cancer cells. The methods may further comprise predicting cancer recurrence in the subject. In some embodiments, peripheral intracellular localization of at least one biomarker (e.g., PFKL, PKKFB4, pGLUT1, etc.) predicts cancer recurrence or indicates recurrent cancer. A biomarker (e.g., PFKL, PKKFB4, pGLUT1, etc.) has peripheral intracellular localization when it is not centrally or homogeneously located throughout the cell but rather the localization is towards the edges of the cell near the cell membrane.

The intracellular localization may be determined using any histochemical analysis well known in the art. Histochemical analyses include but are not limited to, immunohistochemistry or immunostaining, cytochemistry, histopathology, in situ hybridization, and the use of molecular probes. Texts illustrating histochemical techniques include “Histochemical and Immunochemical Techniques: Application to pharmacology and toxicology,” (1991) Bach, P. and Baker, J., eds., Chapman & Hall, New York, N.Y. pp 1-9, and in “Stains and Cytochemical Methods,” (1993) M. A. Hayat, ed., Plenum Press, New York, N.Y., incorporated herein by reference.

In some embodiments, determining intracellular localization of at least one biomarker for cancer recurrence comprises: a) immunostaining the sample with a primary antibody directed to at least one biomarker for cancer recurrence; and b) imaging the sample.

Detecting the primary antibody may be done directly or indirectly. In some embodiments, the primary antibody is detected with a secondary antibody configured to noncovalently attached to the primary antibody. Examples of secondary antibody include anti-mouse, rabbit, bovine, goat, sheep, dog and chicken antibodies. The secondary antibody comprises a detectable label, e.g. a fluorescent tag, a luminescent tag, an enzyme, an enzyme substrate, or a radiolabel covalently attached to the antibody. In some embodiments, the primary antibody is detected by a non-antibody binding protein such as protein G, protein A, protein L, and a lectin which may contain a detectable label as described for the secondary antibody. In some embodiments, primary or secondary antibodies may be suitable antibody fragments (e.g., Fab, Fab′, F(ab′)₂, Fv, scFv, Fd, diabodies, etc.) or other antigen binding elements (e.g., DARPin, anticalin, nanobody, aptamer, affimer, etc.). Alternatively, the primary antibody may contain a detectable label, as described above, or may be modified with another type of label (e.g. biotin) that binds or interacts with a labeled or non-labeled non-antibody binding partner (e.g. streptavidin).

The type of imaging will be dictated by the detectable labels employed. In exemplary embodiments, a secondary antibody is fluorescently labeled and the imaging comprises fluorescence microscopy. In some embodiments, the secondary antibody comprises an enzyme (e.g. peroxidase or alkaline phosphatase) that produces colored products detectable by light microscopy. In some embodiments, the secondary antibody comprises a radioactive label which can be visualized by autoradiography.

The method may further comprise immunostaining for organelles and other cellular structures, e.g., nuclei and cell membranes, using known methods in the art.

In particular embodiments, the intracellular localization of one or both of PFKL and PKKFB4 are analyzed/quantitated/monitored in cell(s). In some embodiments, the intracellular localization of one or more additional biomarkers are also analyzed/quantitated/monitored in cell(s), such as enzymes and transporters involved in the glutathione cycle including, but not limited to, glutamate cysteine ligase catalytic domain (GCLC), glutathione synthetase (GS), cystine-glutamate antiporter (xCT), CD44v9, glutamine uptake transporters ASCT2, ATBO+ and LAT1, leucine uptake (LAT1), gamma-glutamyl transpeptidase (GGT), gamma-glutamyl cysteine (gGC), glucose transporter 1 (GLUT1), glucose 6-phosphate dehydrogenase (G6PD), transketolase (TKT), transketolase-like protein 1 (TKTLP1), RhoA, RhoA with bound GTP and CD74. In some embodiments, peripheral intracellular localization of the biomarkers predicts cancer recurrence or indicates recurrent cancer.

The cancer may be a carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, muscle tissue, ovary, pancreas, prostate, skin, spleen, stomach, testicle, thyroid or uterus. In some embodiments, the cancer is selected from breast cancer, prostate cancer, lung cancer, melanoma, kidney cancer, thyroid cancer, pancreatic cancer, stomach cancer or bladder cancer. The breast cancer may comprise ductal carcinoma in situ of the breast (DICS), lobular carcinoma in situ (LCIS), atypical ductal hyperplasia (ADH), or atypical lobular hyperplasia (ALH). In some embodiments, the cancer recurrence is ipsilateral breast cancer recurrence.

The sample may be any sample which comprises cancer cells, such as a sample from a subject, such as a cancer biopsy or other conventional pathology samples. In some embodiments, the sample comprises a formalin-fixed paraffin-embedded cancer tissue sample or a cancer metastases tissue or cell sample.

The methods may further comprise treating a subject predicted to have cancer recurrence. The treatment or therapeutic regimen may include, but is not limited to, surgery, administration of an inhibitor of enzyme accumulation at the plasma membrane immunotherapy, radiotherapy, administration of a chemotherapeutic agent. In some embodiments, the treatment or therapeutic regimen comprises surgery. In some embodiments, the treatment or therapeutic regimen comprises administration of inhibitors to enzyme or transporter accumulation at plasma membrane. Inhibitors to enzyme accumulation at plasma membrane include, but are not limited to, colchicine, taxol, calmodulin antagonists, anesthetics (e.g. local anesthetics—see Schwartz D, et al, Mol Genet Metab. 2000; 69(2):159-164, incorporated herein by reference in its entirety) and prenylation inhibitors. In some embodiments, the treatment regimen comprises one or more of surgery; administration of inhibitors to enzyme accumulation at plasma membrane; immunotherapy; radiotherapy; and administration of a chemotherapeutic agent.

In some embodiments, a subject predicted to have cancer recurrence is treated with one or more of a chemotherapeutic, immunotherapeutic, radiation, surgery, etc.

In some embodiments, a subject predicted to have cancer recurrence is treated with a suitable chemotherapeutic. In some embodiments, the chemotherapeutic is selected from the group consisting of mitotic inhibitors, alkylating agents, anti-metabolites, intercalating antibiotics, growth factor inhibitors, cell cycle inhibitors, enzyme inhibitors, topoisomerase inhibitors, protein-protein interaction inhibitors, biological response modifiers, anti-hormones, angiogenesis inhibitors, and anti-androgens. Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylomithine (DMFO). Where desired, a subject it treated with one or more commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

In some embodiments, a subject predicted to have cancer recurrence is treated with radiation. In some embodiments, radiation therapy is administered for inhibiting abnormal cell growth or treating a hyperproliferative disorder. Techniques for administering radiation therapy are known in the art. Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

In some embodiments, a subject is treated with an amount of one or more substances selected from anti-angiogenesis agents, signal transduction inhibitors, antiproliferative agents, glycolysis inhibitors, or autophagy inhibitors.

Anti-angiogenesis agents may be selected from agents such as MMP-2 (matrix-metalloproteinase 2) inhibitors, MMP-9 (matrix-metalloprotienase 9) inhibitors, and COX-11 (cyclooxygenase 11) inhibitors. Anti-angiogenesis agents include, for example, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (e.g., MAP-1, MMP-3, MMP-4, MMP-5, MMP-6, MMP-7, MMP-8, MMP-10, MMP-11, MMP-12, and MMP-13). Some specific examples of MMP inhibitors useful in the invention are AG-3340, RO 32-3555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

Tumor cells exhibiting enzyme and transporter trafficking may have high GSH levels and may be able to resist oxidant-mediated chemotherapy and radiotherapy. It is contemplated that inhibition of enzyme and transporter trafficking to the cell periphery in recurrent disease may cause recurrent cancer cells to assume the metabolic properties of non-recurrent cancer cells. In some embodiments, agents inhibiting enzyme accumulation at the plasma membrane are administered to increase the radiosensitivity and chemosensitivity for redox-active drugs. Drug-mediated detachment of glycolytic enzymes from plasma membranes and cytoskeletons (e.g., colchicine, taxol, calmodulin antagonists) have been reported. In some embodiments, DCIS patients exhibiting RhoA and RhoA(GTP) trafficking benefit from prenylation inhibitors, because RhoA's membrane form can undergo prenylation. Thus, in some embodiments, the treatment regimen comprises co-administration of inhibitors of enzyme and transporter accumulation at the plasma membrane and chemotherapy or radiotherapy. In some embodiments, a prenylation inhibitor is administered before treatment to prevent prenylation. In some embodiments, a prenylation inhibitor is administered after treatment to block further prenylation.

In cases of co-administration or selection of more than one treatment regimen, the different therapeutic regimens may be administered together, separately, or subsequently to each other separated by a period of time. For example, the treatment with inhibitors of enzyme and transporter accumulation at plasma membrane may precede any chemotherapy and radiotherapy by a period of time ranging from 1 day to 60 days or surgery may precede administration of inhibitors to enzyme accumulation at plasma membrane, immunotherapy, radiotherapy, or administration of a chemotherapeutic agent.

In those instances when the subject is not predicted to have recurrent cancer, the subject may be monitored and subsequent analysis may be completed during the course of monitoring.

The present disclosure also provides systems (e.g., reagents, computer software, imaging instruments, etc.) for predicting cancer recurrence or distinguishing between recurrent and non-recurrent cancer. The systems may comprise at least one or all of a sample (e.g., positive and/or negative control samples), a primary antibody to a biomarker for cancer recurrence, an imaging instrument (e.g. fluorescence or brightfield microscope), and software configured to determine the intracellular location of the biomarker for cancer recurrence. The description of a sample, biomarkers for cancer recurrence and imaging techniques described elsewhere herein are also applicable to the disclosed system.

The software may be supplied with the systems in any electronic form such as a computer readable device, an internet download, or a web-based portal. The software may be integrated with the imaging instrument to not only determine the intracellular location of the biomarker for cancer recurrence but also predict cancer recurrence. The software may allow a user to view results in real-time, review results of previous samples, and view reports.

The systems can also comprise instructions for using the components of the systems. The instructions are relevant materials or methodologies pertaining to the systems. The materials may include any combination of the following: background information, list of components and their availability information (purchase information, etc.), brief or detailed protocols for using the systems, trouble-shooting, references, technical support, and any other related documents. Instructions can be supplied with the systems or as a separate member component, either as a paper form or an electronic form which may be supplied on computer readable memory device or downloaded from an internet website, or as recorded presentation.

It is understood that the disclosed systems can be employed in connection with the disclosed methods.

### EXPERIMENTAL

Cell metabolism plays an important role in determining cell fate (Refs. 32, 41, 54; incorporated by reference in their entireties). For example, aerobic glycolysis—the catabolism of glucose to lactate in the presence of oxygen—is important in developmental biology, endothelial cell function and adaptive immunity (Refs. 13, 32, 39, 41, 54; incorporated by reference in their entireties). Aerobic glycolysis is also a hallmark of aggressive cancer, and is known as the Warburg effect (Ref. 57; incorporated by reference in its entirety). Cancers have various levels of aggressiveness (Refs. 18, 22, 37; incorporated by reference in their entireties). For example, carcinomas in situ of the thyroid rarely become invasive (Ref. 20; incorporated by reference in its entirety) whereas carcinomas in situ of the bladder frequently recur (Ref. 38; incorporated by reference in its entirety). Several studies have shown that ductal carcinoma in situ of the breast is heterogeneous, and can be indolent or aggressive in nature (Refs. 18, 22, 37; incorporated by reference in its entirety). Experiments were conducted during development of embodiments herein to demonstrate that non-recurrent and recurrent forms of cancer are due to differences in metabolic programming.

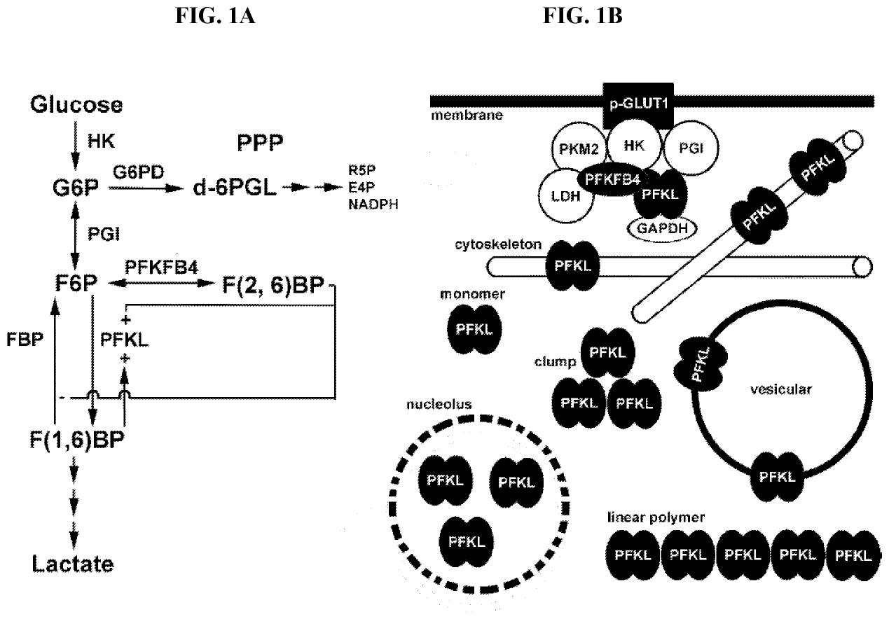

Metabolic programming and tumor cell aggressiveness depend upon glucose transport (Ref. 1). Using DCIS as a model system, experiments have demonstrated that accumulation of phospho-Ser226-GLUT1 near epithelial cell surfaces of cribriform, comedo, papillary, and micropapillary DCIS lesions predicts breast cancer recurrences (Ref. 31; incorporated by reference in its entirety). As breast tissue utilizes facilitated diffusion for glucose uptake (Ref. 65; incorporated by reference in its entirety), glucose transport across the basolateral epithelial surface occurs when the glucose concentration in the tumor interstitial fluid (TIF) is greater than its intracellular level. As TIF glucose levels are reduced compared to normal interstitial fluid (Ref. 25; incorporated by reference in its entirety), intracellular glucose must be rapidly metabolized to maintain a glucose gradient and enable net glucose uptake. Glycolytic rates are regulated by four flux-controlling steps (Ref. 53; incorporated by reference in its entirety). Phosphofructokinases type 1 (PFKT), which includes PFKL, PFKM, and PFKP, catalyze a flux controlling step by converting F6P to F(1, 6)BP (FIG. 1A). PFKL is activated by its product, F(1, 6)BP (46). PFKIs are activated by AMP, PLC-mediated phosphorylation, F6P, and F(2, 6)BP (56). F(2, 6)BP, the most potent activator of PFKIs, is produced by PFKFB4, a type 2 PFK. Both PFKL and PFKFB4 enhance glycolytic activity, and have been shown to correlate with aggressive cancers (Refs. 35, 40, 47; incorporated by reference in their entireties). Moreover, experimental manipulations of PFKL or PFKFB4 levels promote parallel changes in animal outcomes (Refs. 16, 47, 62; incorporated by reference in their entireties). In contrast, fructose 1,6-bisphosphatase (FBP) catalyzes F6P synthesis from F(1, 6)BP (FIG. 1A) and diminishes the aggressive phenotype (Ref. 34; incorporated by reference in its entirety). Hence, PFKL and PFKFB4 contribute to heightened glycolysis and tumor aggressiveness.

In addition to biochemical factors, enzyme activity is also influenced by physical properties such as environment, binding partners, and clustering. Factors such as pH, potassium concentration, and redox conditions may impact enzyme activity (Ref. 44; incorporated by reference in its entirety). Because enzyme clustering reduces the distances between consecutive steps in a biochemical pathway, it accelerates product formation (Ref. 7, 11, 50; incorporated by reference in their entireties). In addition to reducing the time between consecutive enzymatic steps, enzyme proximity improves product formation because insoluble or reactive intermediates and those intermediates intersecting other pathways may not exhibit product formation in solution. Biological strategies to regulate enzyme location include aggregation, oligomerization, and concentration within organelles or along membranes. PFKL locations include: plasma membranes, microfilaments, linear polymers, cytoplasmic monomers and clumps, intracellular membranes, and nucleoli (FIG. 1B) (Ref. 30, 55, 58; incorporated by reference in their entireties). PFK type 1 may interact with itself, other PFK1 isotypes, microfilaments, and plasma membrane components including metabolons and caveolin (Ref. 30, 52; incorporated by reference in their entireties). PFKFB4 is known to befound in nucleoli (Ref. 55; incorporated by reference in its entirety). PFK undergoes intracellular redistribution (Refs. 3, 59; incorporated by reference in their entireties), including vesicular transport (Ref. 63; incorporated by reference in its entirety). The heterogeneity of PFK assembly is expressed by tumor cells in vitro. In addition to metabolons, PFKL has been found to assemble into large clusters and filaments in tumor cells, but not in “normal” breast cells (Ref. 30; incorporated by reference in its entirety). Similarly, PFKL clusters are found in hepatocarcinoma cells exposed to hypoxia (Ref. 27; incorporated by reference in its entirety). Enzyme trafficking may influence enzyme locations, enzyme activities and physiological outputs (Ref. 29; incorporated by reference in its entirety).

When studying the localization of PFKL and PFKFB4, the markers could be present in multiple assembly states at various locations; it is impossible to score all of the variables by hand. Machine learning, by contrast, uses algorithms to solve classification problems by pattern recognition. Experiments were conducted during development of embodiments herein to using machine learning to predict patient outcomes based upon PFKL and PFKFB4 labeling patterns. A previous study of phospho-GLUT1-labeled tissue samples could not accurately predict patient outcomes using images of solid DCIS lesions (Ref. 31; incorporated by reference in its entirety). However, experiments conducted during development of embodiments herein demonstrated that patients exhibiting breast cancer recurrences release PFKFB4 and PFKL from epithelial cell nucleoli, which then localize to the cell periphery. Images of PFKL, PFKFB4, and phospho-GLUT1 labeling in combination with machine learning accurately predicts 10-year patient outcomes of all DCIS samples. This demonstrates that spatial changes in PFKL, PFKFB4, and phospho-GLUT1 distributions: participate in cancer recurrences, and provide a prognostic test for identifying patients at high and low risk for cancer recurrences.

### Methods

### Patient Samples

DCIS patient samples. DCIS samples were studied from 101 women (51 non-recurrent, 50 recurrent) who were followed for at least 10 years. For all DCIS samples, no evidence of lymph node involvement was noted. No evidence of invasive cancer was present. Tissue samples were from partial or total mastectomies of women aged 37-80 years after informed consent was obtained. Patients had no previous or concurrent cancer. FFPE pathology samples were obtained from the St. Louis Breast Tissue Registry (St. Louis, MO) and Beaumont Hospital (Royal Oak, MI). This blinded tissue sourcing strategy was used to ensure that laboratory personnel did not have access to electronic medical records of patients whose samples were under study. The use of human material was in accordance with the Declaration of Helsinki on the use of material in scientific research. All experiments were approved by the University of Michigan IRB (number HUM000044189).

Metastatic breast cancer and normal adjacent tissue samples. Samples of breast cancer metastases were obtained from the NDRI (National Disease Research Interchange; Philadelphia, PA). Patients were 59-75 years of age. The primary tumors were characterized as: invasive mammary carcinoma lobular type, infiltrating ductal carcinoma, and inflammatory breast cancer (N=5). Metastasis to the omentum was found to best illustrate protein localization within metastatic cells. Normal adjacent tissue (NAT) was acquired from NDRI (N=3). These samples were from patients aged 47-72 years who were diagnosed with invasive ductal carcinoma or DCIS.

### Microscopy

Immunofluorescence of tissue sections. FFPE samples were cut into 5 μm thick sections. Sections were de-paraffinized and re-hydrated by sequential incubation in a graded ethanol series. After rehydration in PBS with 0.02% Triton X-100 (Thermo-Fisher Sci.), sections were subjected to heat-mediated antigen retrieval in 10 mM citric acid buffer, pH 6.0. Sections were blocked using a blocking solution (10% dried milk in PBS and/or 1% BSA in Tris-buffered saline plus 0.10% Tween-20) for 2 hr. at room temperature. After blocking procedures, sections were incubated with antibody (Table 1) diluted in 1% BSA in PBS overnight at 4° C. After incubation, the sections were washed with PBS. Finally, the sections were incubated with a fluorescent secondary antibody for 1 hr., washed with PBS, and then mounted in Prolong Diamond Antifade medium (Thermo-Fisher Sci., Waltham, MA).

TABLE 1

| | | | | | | |
|---|---|---|---|---|---|---|
| Antibody | Species of | | Catalog | | Vial | Test |
| target | antibody | Provider | Number | Lot Number | Concentration | Concentration |

Primary Antibodies

| | | | | | | | | |
|---|---|---|---|---|---|---|---|---|
| Phospo- | Rabbit | Millipore | ABN991 | 3426909 | 1 | mg/mL | 2 | μg/mL |
| GLUT1 | | | | | | | | |
| PFKL | Mouse | Santa Cruz | sc-393713 | 10817 | 0.2 | mg/mL | 2.2 | μg/mL |
| PFKFB4 | Rabbit | Abcam | ab137785 | GR237390-39 | 1 | mg/mL | 2.2 | μg/mL |
| PFKM | Rabbit | Proteintech | 55028-1-AP | 80018 | 0.574 | mg/mL | 10 | μg/ml |
| PFKP | Mouse | Abcam | Ab119796 | GR3287100-8 | 0.527 | mg/mL | 6.7 | μg/ml |
| CD31 | Mouse | Abcam | ab197770 | GR237390-39 | 1 | mg/mL | 2.2 | μg/mL |
| Histone | Mouse | Abcam | ab229914 | GR3285908-3 | 0.511 | mg/mL | 2.5 | μg/ml |
| H2A.X | | | | | | | | |
| Nucleolin | Rabbit | Abcam | ab86727 | GR109659-1 | 1 | mg/ml | 5 | μg/ml |

Secondary Antibodies

| | | | | | | | | |
|---|---|---|---|---|---|---|---|---|
| Mouse IgG | Goat | Thermo- | A11029 | 2120125 | 2 | mg/mL | 20 | μL/mL |
| | | Fisher | | | | | | |
| Rabbit IgG | Donkey | Thermo- | A10042 | 2136776 | 2 | mg/mL | 20 | μL/mL |
| | | Fisher | | | | | | |

Imaging. Fluorescence microscopy was performed (Refs. 14, 15; incorporated by reference in their entireties) using a Nikon TE2000-U inverted microscope (Nikon, Melville, NY) with a 20× (0.55NA) objective, 1.5× optivar and a back-illuminated Andor iXon electron-multiplying charge-coupled device (EMCCD) camera (Model DV-888; Andor Technology, Belfast, Northern Ireland). Images were captured and processed with Metamorph software (Molecular Devices, Downingtown, PA). To reduce shot noise, each micrograph was an average of 10-15 images, with each image acquired for 0.2 sec. To reduce read noise, the EMCCD chip was cooled to −95° C. Typical camera settings were: multiplication gain, 100; vertical shift speed, 3.04 msec./pixel and 14-bit digitization at 10 MHz. Micrographs were evaluated and auto-scaled using ImageJ software.

### Data Analysis

Statistical analysis of categorical data. The statistical significance of comparisons of biomarker locations was assessed using contingency tables. Two-by-two contingency tables were constructed using the categorical variables of peripheral or non-peripheral staining and patient outcomes. Tests were performed with the “N−1” chi-squared test (Ref. 10; incorporated by reference in its entirety).

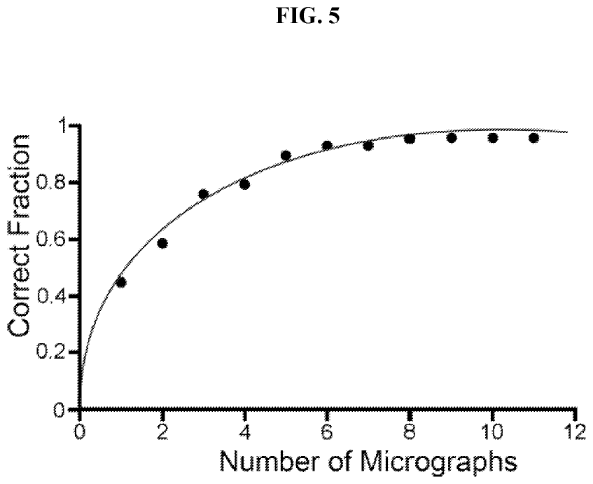

Computed outcome prediction. As the frequency of cancer promoting cells is not known in DCIS samples from patients reporting recurrences, multiple low power micrographs were obtained of recurrent tissue samples. Each micrograph was evaluated for recurrent/non-recurrent predicted outcomes. To estimate the number of micrographs necessary for a correct prediction, 29 consecutive patients reporting a recurrence were assessed. For each of the patients, the number of micrographs required to reach the first micrograph yielding the anticipated recurrence prediction was determined. The data are plotted in FIG. 5. At least six micrographs are required to ensure that at least one recurrent cancer prediction is made each recurrent sample. On the basis of these findings, 10 or more micrographs were obtain of each section for computational analysis.

The Custom Vision application of Microsoft's (Redmond, WA) Azure Cognitive Services platform was used for machine outcome predictions. Custom Vision is a state-of-the-art computer vision application. This software tool was deployed as a multiclass (tags: recurrent or non-recurrent) and general domain problem.

The computer was trained with micrographs of PFKL and PFKFB4. Machine training was based on tissues exhibiting peripheral or non-peripheral labeling patterns. This approach minimized the introduction of confounding errors by excluding known false negatives and apparent false positives (recurrence-free patients with peripheral protein labeling) from the training dataset. Micrographs categorized as indeterminant, those with poor focus, and those containing artifacts such as tissue section folds, were not used for training. Limited dataset size is a common problem in medical machine vision applications. To deflect this issue, image augmentation was used (Ref. 51; incorporated by reference in its entirety’). Image mirroring was used to increase the dataset's size. The performance of the computer model was assessed by calculating precision and recall. The precision (or positive predictive value) is:

$$Precision = TP / ( TP + FP )$$

where TP=true positives and FP=false positives. The recall is defined as:

$$Recall = TP / ( TP + FN )$$

where FN=false negatives. Data were evaluated using precision-recall curves, which plot these variables across many threshold values. Precision-recall curves are much less sensitive to differences in the numbers of patients in each group than receiver operating characteristic plots (Ref. 49; incorporated by reference in its entirety). The cut-point was typically used at a threshold of 50%. The probability of outcome for each micrograph undergoing computer-assisted diagnosis as recurrent or non-recurrent was generally 99.9 or 100%.

### Results

### PFK Labeling Patterns in DCIS Lesions

Experiments were conducted during development of embodiments herein to compare PFK labeling patterns of tissue samples from DCIS patients who did not experience a cancer recurrence to those who did experience a recurrence. Phospho-Ser226-GLUT1 accumulates at the periphery of epithelial cells within cribriform, comedo, papillary, and micropapillary DCIS lesions from patients who will experience a recurrence (Ref. 31; incorporated by reference in its entirety). Phosphorylation of Ser226 increases GLUT1's Vmax (Ref. 33; incorporated by reference in its entirety). GLUT1 facilitates glucose movement down its concentration gradient, and intracellular glucose breakdown is necessary to maintain an inward glucose flux. As F(1, 6)BP formation is a flux-controlling step of glycolysis,

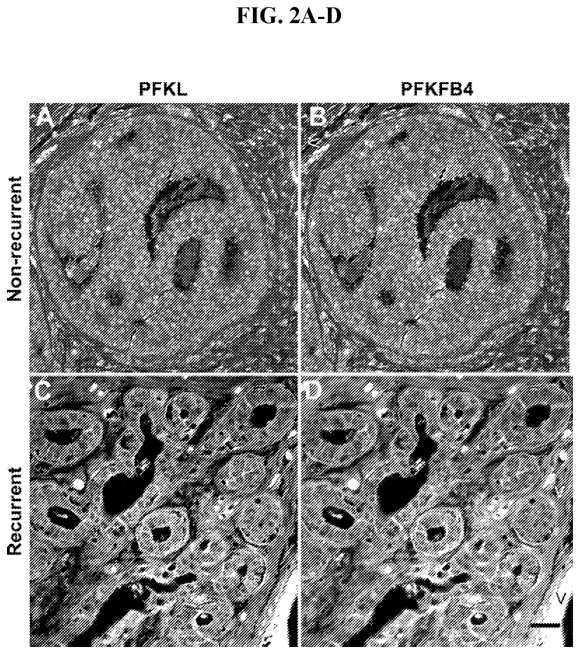

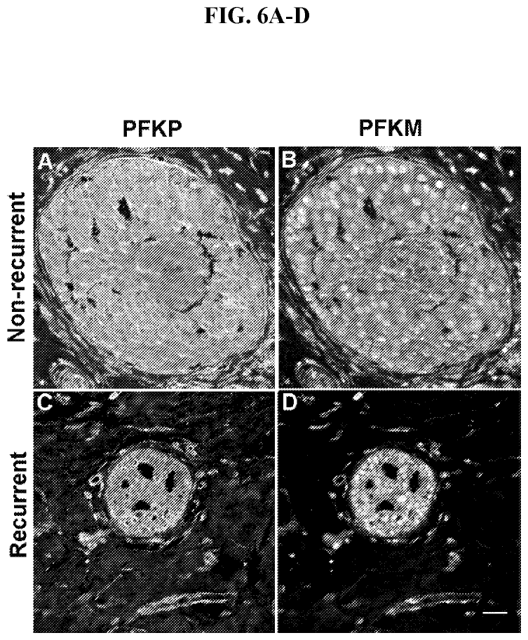

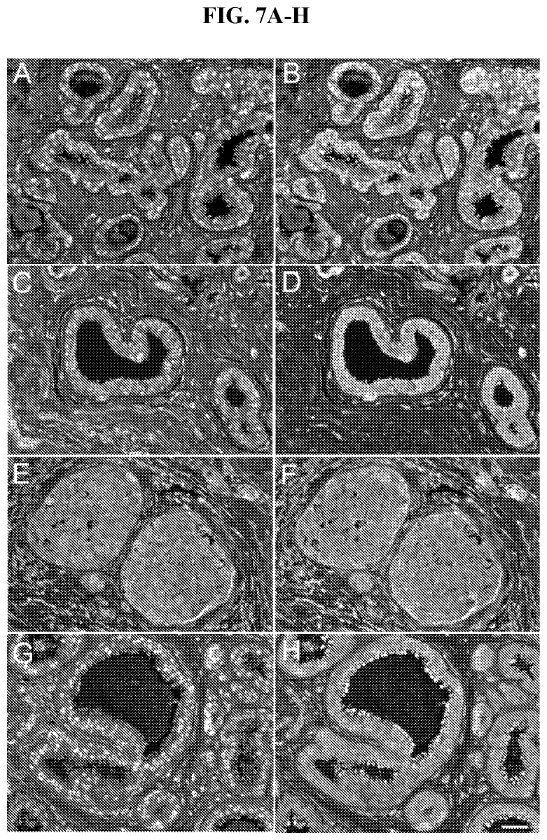

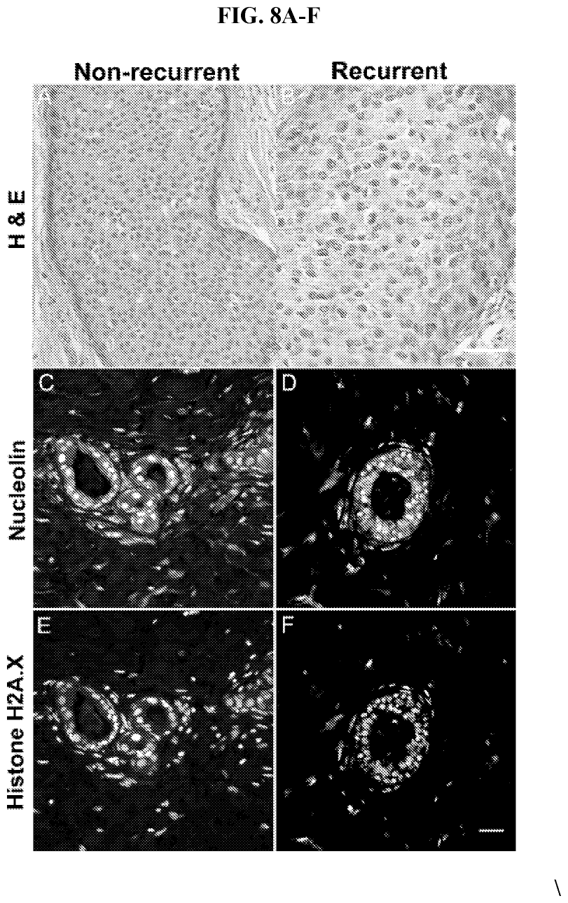

To test whether intracellular PFK isotype trafficking accompanies cancer recurrences, experiments were conducted during development of embodiments herein to study the spatial features of PFKs using a panel of monoclonal antibodies (Table 1). FIG. 6A, C shows that PFKP is peripherally located in samples from women who will and will not experience a subsequent cancer recurrence. PFKM was centrally located in ductal epithelial cells of biopsies of both recurrent and non-recurrent DCIS patients (FIG. 6B, D). In contrast, PFKL was found to be centrally located in cells of non-recurrent patients (FIG. 2A), but peripherally located in cells of patients who will experience recurrent cancer (FIG. 2C). PFKL images because they exhibited a significant re-distribution event in samples from patients exhibiting recurrences. Additionally, PFKL and PFKFB4 were chosen because their increased expression levels correlate with reduced patient survival times (Refs. 35, 40, 47; incorporated by reference in their entireties). FIG. 2B, D shows fluorescence micrographs of DCIS samples stained with antibodies directed against PFKFB4. In samples from women who did not report a recurrence, PFKL and PFKFB4 were observed in the nucleolar region (FIG. 2A, B). Additional examples of PFKL and PFKFB4 labeling of non-recurrent DCIS lesions are shown in FIG. 7. PFKL and PFKFB4 have been reported to be nucleolar proteins (Ref. 55; incorporated by reference in its entirety). FIG. 2A, B suggest that the staining patterns of PFKL and PFKFB4 match those of nucleoli. This interpretation is consistent with H&E staining patterns of DCIS samples (FIG. 8A, B). DCIS tissue was also tagged with anti-nucleolin and anti-H2A.X antibodies, with similar results (FIG. 8C-F). Thus, PFKL and PFKFB4 appear to reside in the nucleolar region of epithelial cells of DCIS samples from patients who will not experience a subsequent recurrence.

The properties of PFKL and PFKFB4 was evaluated in DCIS samples of patients who will exhibit a cancer recurrence. For DCIS patients experiencing cancer recurrences, PFKL and PFKFB4 were generally not found in the nucleus, but were localized to the cell periphery. The examples of FIGS. 2C and D illustrate peripheral PFKL and PFKFB4 labeling, respectively. Changes in PFK distribution could mediate regulatory trafficking of PFKL and PFKFB4 by altering enzyme location and glycolytic activity during acquisition of the recurrent cancer phenotype.

The peripheral or non-peripheral distributions of PFKL and PFKFB4 for each DCIS patient were scored, then assembled into Table 2. To document the statistical significance of these observations, the intracellular locations of PFKL and, independently, PFKFB4 were treated as categorical variables (peripheral or non-peripheral) in statistical analyses. A single micrograph of a patient exhibiting peripheral biomarker labeling was sufficient to define that patient as exhibiting peripheral labeling. Although the peripheral accumulation of PFKL or PFKFB4 could vary from patient-to-patient, both contingency tables yielded P<0.0001. It should be noted that five cases of contralateral recurrences and one unreadable sample were not included in these calculations. These population findings suggest that the relocation of PFKs within pre-invasive epithelial cells is highly significant.

TABLE 2
Cross-Validation Studies on the Performance of a
Computer Model using PFKL and PFKFB4 Labeling¹⁻³

| | | | Average | |
|---|---|---|---|---|
| Outcome | Precision | Recall | Performance | N (images) |
| Recurrent cancer | 100.0% | 84.9% | 95.8% | 373 |
| Non-recurrent cancer | 89.0% | 97.3% | 92.8% | 362 |

¹The terms precision and recall are parameters used for classification models in machine learning. The precision and recall are defined in the Methods.
²N is the number of micrographs.
³A total of 78 patients were used to construct this model.

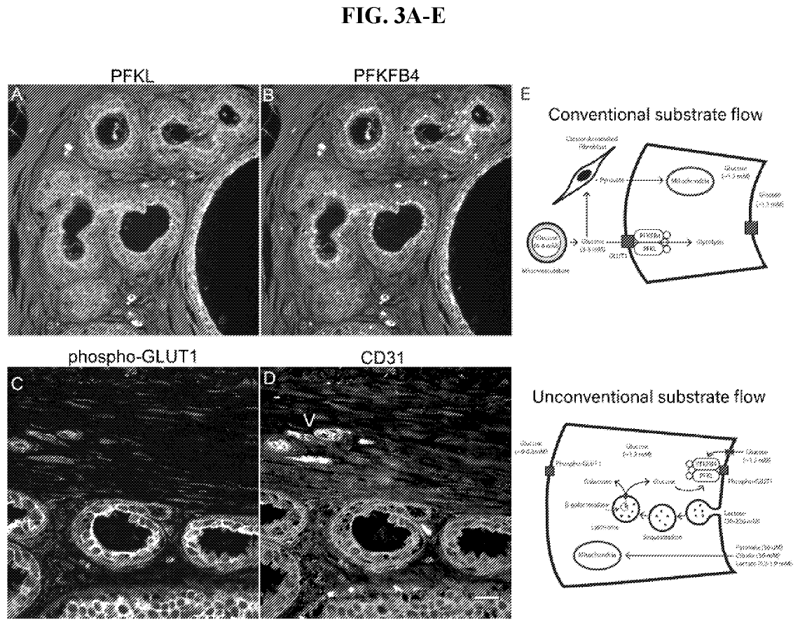

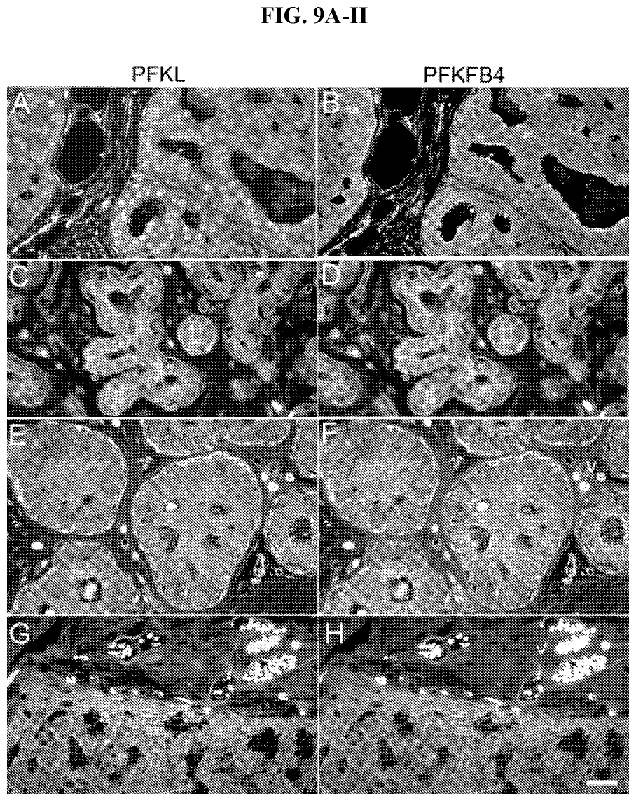

Heterogeneity of PFKL and PFKFB4 disposition in samples from recurrent patients To further illustrate the heterogeneity of labeling patterns for samples from patients exhibiting recurrences, FIG. 9 is shown. Although PFKL was frequently found at the cell periphery, it may also be found in the nucleolus, and cytoplasm (FIG. 3C; FIGS. 9A, C, E, and G). PFKFB4 was often found at the cell periphery, but could also be heterogeneously distributed (FIG. 2D; FIG. 9C, D). This heterogeneity may reflect different stages of lesion development in patients who will experience a recurrence.

Although peripheral labeling is typically uniform, specific biomarker clustering at the apical surface are observed. For example, FIG. 3A, B shows dramatic PFKL and PFKFB4 accumulation at the apical surface of epithelial cells (2 of 50 patients reporting recurrences). Apical clustering is unexpected because: 1) the basolateral surface has access to glucose from the cardiovascular system and 2) PFK influences glucose uptake and catalyzes a flux-controlling glycolytic step. As glucose, lactose and other carbon sources are released into the lumen by normal epithelial cells of women under 50 (Ref. 45; incorporated by reference in its entirety), it is possible that some pre-invasive cells may adapt to low interstitial glucose levels by harvesting carbohydrates from the lumen. To determine if other key glycolytic steps traffic to the apical surface, phospho-Ser-226-GLUT1 was examined (FIG. 3C); this biomarker was apically expressed in samples that also demonstrated apical PFKL/PFKFB4 labeling. This apically-enhanced labeling was unlikely to be a preparation artifact because it was not found in all ducts. For example, solid DCIS ducts from this patient did not show enhanced apical labeling. To examine if redundant membranes such as microvilli and membrane folds could account for enhanced apical labeling, these cells were also stained with anti-CD31, which is found on the plasma membranes of ductal epithelial cells (Ref. 43; incorporated by reference in its entirety). In contrast to phospho-Ser226-GLUT1, PFKL and PFKFB4, CD31 was found at both the basolateral and apical surfaces at comparable levels (FIG. 3D). Thus, it appears that epithelial cells within DCIS lesions may obtain energy from carbohydrates in milk by transporting and catabolizing glucose at their apical surfaces.

### PFKL and PFKFB4 in Breast Cancer Metastasis

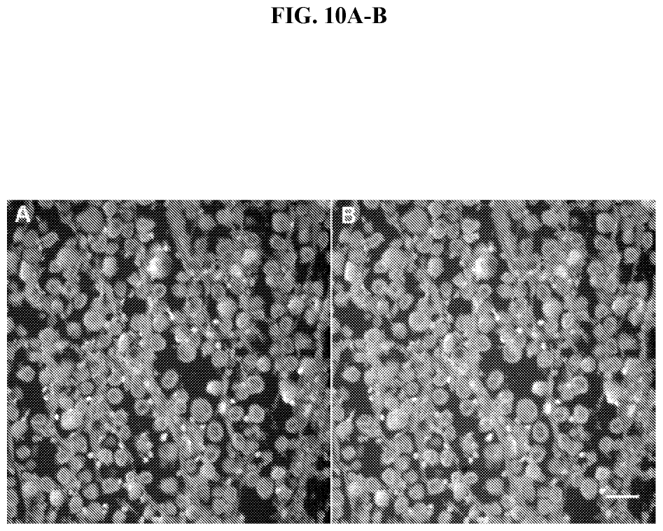

PFKL and PFKFB4 labeling patterns of samples of breast cancer metastases were examined. FIGS. 10A and B show that PFKL and PFKFB4 are found at the periphery of metastatic cells. Peripheral PFK labeling patterns in metastatic breast cancer cells parallel those of ductal epithelial cells of DCIS patients destined to experience recurrences.

### Prognostic Utility of PFKL and PFKFB4 in DCIS

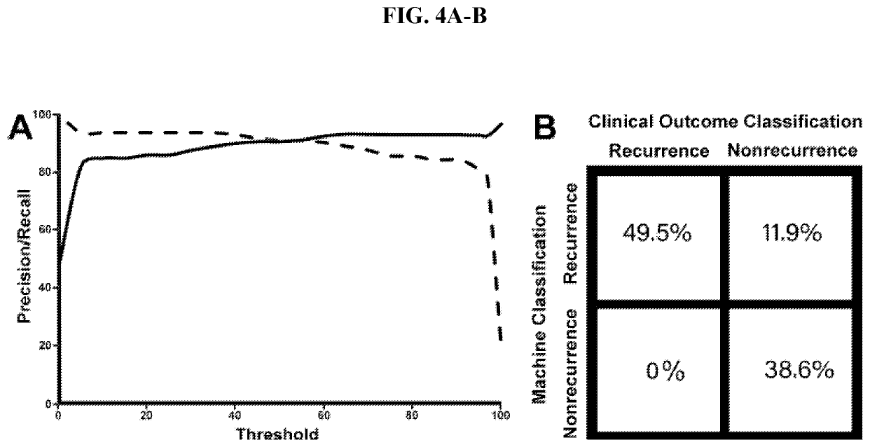

To determine if these markers could predict individual patient outcomes, machine learning tests were performed. Machine learning was used to make outcome predictions because it provides results superior to visual observations (Ref. 31; incorporated by reference in its entirety), due to its ability to identify multiple predictive image elements. Computational models were developed to predict patient outcomes using PFK labeling patterns of patients in the recurrent or non-recurrent classes on the Azure cloud platform. As all patients had partial or full mastectomies, it is impossible to know if the original lesion would have led to cancer. Therefore, machine training was performed as previously described using (peripheral=recurrence and not peripheral=no recurrence) for image selection while excluding micrographs inconsistent with this standard (Ref. 31; incorporated by reference in its entirety). Optimal findings were obtained when the computer was simultaneously trained using images of PFKL and PFKFB4-labeled tissue sections, presumably due to the increase in sample size and similarity of spatial changes. Cross-validation tests of the dataset yielded average performances of 95.8% for recurrent cancers and 92.8% for non-recurrent cancers. The precision and recall of the PFKL/PFKFB4 computational model are plotted as a function of threshold in FIG. 4A and details are summarized in Table 1. With this computer model, individual patients were evaluated using micrographs that were withheld from the training process. Outcomes were predicted using a threshold (a computed probability of outcome) of 50%. One micrograph exceeding this threshold was sufficient to classify the patient's outcome as a recurrence.

Using DCIS lesions of all morphologies, the PFKL and PFKFB4 computer outcome predictions were correct in 92% of the cases, with 2% false positives and 5% false negatives. Using data concerning phospho-GLUT1 accumulation in the plasma membrane's vicinity for all DCIS morphologies, some of which were recently reported (Ref. 31; incorporated by reference in its entirety), it was found that the outcomes were correct in 80% of the cases, with 11% false positives and 9% false negatives. The machine test correctly identified 88.1% of the cases, with 11.9% false positives and 0% false negatives for our study population (FIG. 4B). This is an optimal experimental finding because false negatives must be avoided for clinical use.

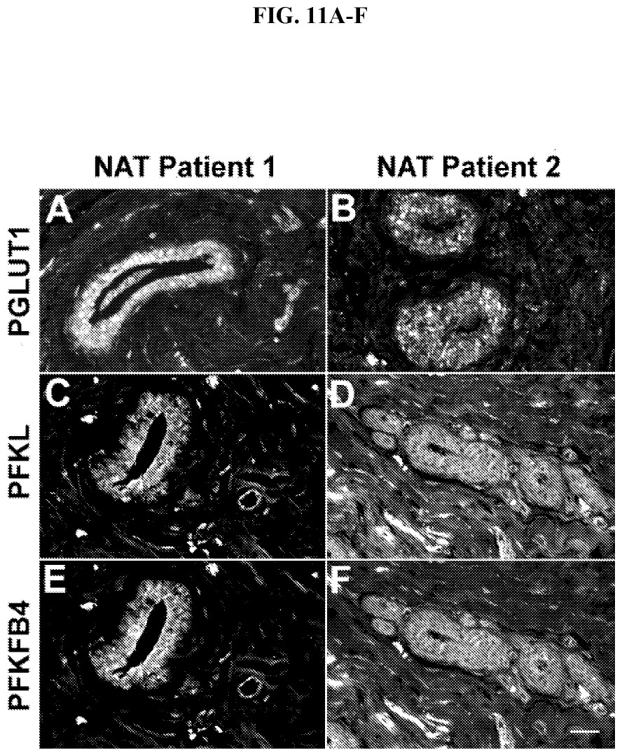

Using normal adjacent tissue (NAT) of breast cancer patients (DCIS and IDC), tissue sections were stained with anti-phospho-Ser226-GLUT1, anti-PFKL and anti-PFKFB4. FIG. 11 shows fluorescence micrographs of PFKL, PFKFB4 and phospho-GLUT1-stained NAT sections. These samples often exhibited non-peripheral biomarker aggregates and peripheral staining. To provide a computational test of these biomarker patterns, machine learning tests were performed of NAT using the above computer models. Micrographs of phospho-Ser226-GLUT1, PFKL, and PFKFB4-labeled NAT predicted cancer recurrences for all NAT patients, but not for all sections or micrographs. In most cases, the diagnostic probability was 99.9-100%.

### REFERENCES

The following references, some of which are also cited above by number, are herein incorporated by reference in their entireties.
1. Adekola K, Rosen S T, Shanmugam M. Glucose transporters in cancer metabolism. Curr Opin Oncol. 24(6):650-4, 2012.
2. An S, Kumar R, Sheets E D, Benkovic S J. Reversible compartmentalization of de novo purine biosynthetic complexes in living cells. Science. 320(5872):103-6, 2008.
3. Attanasio F, Caldieri G, Giacchetti G, van Horssen R, Wieringa B, Buccione R. Novel invadopodia components revealed by differential proteomic analysis. Eur J Cell Biol. 90(2-3), 115-27, 2011.
4. Aran D, Camarda R, Odegaard J, Paik H, Oskotsky B, Krings G, Goga A, Sirota M, Butte A J. Comprehensive analysis of normal adjacent to tumor transcriptomes. Nat Commun. 8(1):1077, 2017.
5. Balmain A. The critical roles of somatic mutations and environmental tumor-promoting agents in cancer risk. Nat Genet. 52(11):1139-1143, 2020.
6. Ballard O, Morrow A L. Human ilk composition: nutrients and bioactive factors. Pediatr Clin North Am. 60(1):49-74, 2013.
7. Bauler P, Huber G, Leyh T, McCammon J A. Channeling by proximity: the catalytic advantages of active site colocalization using Brownian dynamics. J Phys. Chem.

### Lett. 1:1332-1335, 2010.

8. Boisvert F M, van Koningsbruggen S, Navascues J, Lamond A I. The multifunctional nucleolus. Nat Rev Mol Cell Biol. 8(7):574-85, 2007.
9. Bosdriesz E, Wortel M T, Haanstra J R, Wagner M J, de la Torre Cortes P, Teusink B. Low affinity uniporter carrier proteins can increase net substrate uptake rate by reducing efflux. Sci Rep. 8(1):5576, 2018.
10. Campbell I. Chi-squared and Fisher-Irwin tests of two-by-two tables with small sample recommendations Stat. Med. 26: 3661-75, 2007.
11. Castellana M, Wilson M Z, Xu Y, Joshi P, Cristea I M, Rabinowitz J D, Gitai Z, Wingreen N S. Enzyme clustering accelerates processing of intermediates through metabolic channeling. Nature Biotechnol. 2014; 32: 1011-8.
12. Cheatham B, Vlahos C J, Cheatham L, Wang L, Blenis J, Kahn C R. Phosphatidylinositol 3-kinase activation is required for insulin stimulation of pp70 S6 kinase, DNA synthesis, and glucose transporter translocation. Mol Cell Biol. 14(7):4902-11, 1994.
13. Cheng S C, Quintin J, Cramer R A, Shepardson K M, Saeed S, Kumar V, Giamarellos-Bourboulis E J, Martens J H, Rao N A, Aghajanirefah A, Manjeri G R, Li Y, Ifrim D C, Arts R J, van der Veer B M, Deen P M, Logie C, O'Neill L A, Willems P, van de Veerdonk F L, van der Meer J W, Ng A, Joosten L A, Wijmenga C, Stunnenberg H G, Xavier R J, Netea M G. mTOR- and HIF-1α-mediated aerobic glycolysis as metabolic basis for trained immunity. Science. 345(6204):1250684, 2014.
14. Clark A J, Petty H R. Protocol for biomarker ratio imaging microscopy with specific application to ductal carcinoma in situ of the breast. Frontiers in Cell and Developmental Biology 4: 120, 2016.
15. Clark A J, Petty H R. Identification of lesion subtypes in biopsies of ductal carcinoma in situ of the breast using biomarker ratio imaging microscopy. Scientific Rep. 6:27039, 2016.
16. Dasgupta S, Rajapakshe K, Zhu B, Nikolai B C, Yi P, Putluri N, Choi J M, Jung S Y, Coarfa C, Westbrook T F, Zhang X H, Foulds C E, Tsai S Y, Tsai M J, O'Malley B W. Metabolic enzyme PFKFB4 activates transcriptional coactivator SRC-3 to drive breast cancer. Nature. 556(7700):249-254, 2018.
17. Emmott E, Hiscox J A. Nucleolar targeting: the hub of the matter. EMBO Rep. 10(3):231-8, 2009.
18. Esserman L J, Thompson I M, Reid B. Overdiagnosis and overtreatment in cancer: An opportunity for improvement. JAMA 310:797-798, 2013.
19. Faulkner A, Chaiyabutr N, Peaker M, Carrick D T, Kuhn N J. Metabolic significance of milk glucose. J Dairy Res. 48(1):51-6, 1981.
20. Folkman J, Kalluri R. Cancer without disease. Nature. 427(6977):787, 2004.
21. Gay M C L, Koleva P T, Slupsky C M, Toit E D, Eggesbo M, Johnson C C, Wegienka G, Shimojo N, Campbell D E, Prescott S L, Munblit D, Geddes D T, Kozyrskyj A L. InVIVO LactoActive Study Investigators. Worldwide variation in human milk metabolome: indicators of breast physiology and maternal lifestyle? Nutrients. 10(9):1151, 2018.
22. Harding C, Pompei F, Burmistrov D, Welch H G, Abebe R, Wilson R. Breast cancer screening, incidence and mortality across US counties. JAMA Intern. Med. 175, 1483-1489, 2015.
23. Harris B R E, Zhang Y, Tao J, Shen R, Zhao X, Cleary M P, Wang T, Yang D Q. ATM inhibitor KU-55933 induces apoptosis and inhibits motility by blocking GLUT1-mediated glucose uptake in aggressive cancer cells with sustained activation of Akt. FASEB J 35(4):e21264 2021.
24. Hwang E S. The impact of surgery on ductal carcinoma in situ outcomes: the use of mastectomy. J Natl Cancer Inst Monogr. 2010(41):197-9, 2010.
25. Jain R K. Transport of molecules in the tumor interstitium: a review. Cancer Res. 47(12):3039-51, 1987.
26. Jiang P, Du W, Wang X, Mancuso A, Gao X, Wu M, Yang X. p53 regulates biosynthesis through direct inactivation of glucose-6-phosphate dehydrogenase. Nat Cell Biol. 13(3):310-6, 2011.
27. Jin M, Fuller G G, Han T, Yao Y, Alessi A F, Freeberg M A, Roach N P, Moresco J J, Karnovsky A, Baba M, Yates J R 3rd, Gitler A D, Inoki K, Klionsky D J, Kim J K. Glycolytic enzymes coalesce in g bodies under hypoxic stress. Glycolytic enzymes coalesce in G bodies under hypoxic stress. Cell Rep. 20(4):895-908, 2017.
28. Keyser E A, Staat B C, Fausett M B, Shields A D. Pregnancy-associated breast cancer. Rev Obstet Gynecol. 5(2):94-9, 2012.
29. Kindzelskii A L, Huang J B, Chaiworapongsa T, Fahmy R M, Kim Y M, Romero R, Petty, HR. Pregnancy alters glucose-6-phosphate dehydrogenase trafficking, cell metabolism and oxidant release of maternal neutrophils. J Clin. Invest. 110:1801-1811, 2002.
30. Kohnhorst C L, Kyoung M, Jeon M, Schmitt D L, Kennedy E L, Ramirez J, Bracey S M, Luu B T, Russell S J, An S. Identification of a multienzyme complex for glucose metabolism in living cells. J Biol Chem. 292(22):9191-9203, 2017.
31. Kraft A M, Petty H R. Spatial locations of certain enzymes and transporters within preinvasive ductal epithelial cells predict human breast cancer recurrences. Am J Physiol Cell Physiol. 319(5):C910-C921. 2020.
32. Krejci A, Tennessen J M. Metabolism in time and space—exploring the frontier of developmental biology. Development. 144(18):3193-3198, 2017.
33. Lee E E, Ma J, Sacharidou A, Mi W, Salato I V K, Nguyen N, Jiang Y, Pascual J M, North P E, Shaul P W, Mettlen M, Wang R C. A protein kinase C phosphorylation motif in GLUT1 affects glucose transport and is mutated in GLUT1 deficiency syndrome. Mol. Cell 58:845-53, 2015.
34. Li B, Qiu B, Lee D S, Walton Z E, Ochocki J D, Mathew L K, Mancuso A, Gade T P, Keith B, Nissim I, Simon M C. Fructose-1,6-bisphosphatase opposes renal carcinoma progression. Nature. 513(7517):251-5, 2014.
35. Li L, Li L, Li W, Chen T, Bin Zou, Zhao L, Wang H, Wang X, Xu L, Liu X, Wang D, Li B, Mak T W, Du W, Yang X, Jiang P. TAp73-induced phosphofructokinase-1 transcription promotes the Warburg effect and enhances cell proliferation. Nat Commun. 9(1):4683, 2018.
36. Liao M, Liao W, Xu N, Li B, Liu F, Zhang S, Wang Y, Wang S, Zhu Y, Chen D, Xie W, Jiang Y, Cao L, Yang B B, Zhang Y. LncRNA EPB41L4A-AS1 regulates glycolysis and glutaminolysis by mediating nucleolar translocation of HDAC2. EBioMedicine. 41:200-213, 2019.
37. Marshall E. Breast cancer. Dare to do less. Science 343:1454-1456, 2014.
38. Meijer R P, van Onna I E, Kok E T, Bosch R. The risk profiles of three clinical types of carcinoma in situ of the bladder. BJU Int. 108(6):839-43, 2011.
39. Mertens S, Noll T, Spahr R, Kritzfeldt A, Piper H M. Energetic response of coronary endothelial cells to hypoxia. Am J Physiol. 258(3 Pt 2):H689-94, 1990.
40. Minchenko O H, Ochiai A, Opentanova I L, Ogura T, Minchenko D O, Caro J, Komisarenko S V, Esumi H. Overexpression of 6-phosphofructo-2-kinase/fructose-2,6-bisphosphatase-4 in the human breast and colon malignant tumors. Biochimie. 87(11):1005-1010, 2005.
41. Miyazawa H, Aulehla A. Revisiting the role of metabolism during development. Development. 145(19):dev131110, 2018.
42. Montanaro L, Trere D, Derenzini M. Nucleolus, ribosomes, and cancer. Am J Pathol. 173(2):301-10, 2008.
43. Ortiz-Hidalgo C, Torres J E, Cuesta-Mejias T, Mendoza-Ramón H. CD31 with strong membrane-based immunoreactivity in ductal carcinoma of the breast. Appl Immunohistochem Mol Morphol. 8(4):334-5, 2000.
44. Paetkau V, Lardy H A. Phosphofructokinase. Correlation of physical and enzymatic properties. J Biol Chem. 242(9):2035-42, 1967.
45. Petrakis N L, Lim M L, Miike R, Lee R E, Morris M, Lee L, Mason L. Nipple aspirate fluids in adult nonlactating women—lactose content, cationic Na+, K+, Na+/K+ ratio, and coloration. Breast Cancer Res Treat. 13(1):71-8, 1989.
46. Reinhart G D, Lardy H A. Rat liver phosphofructokinase: kinetic activity under near-physiological conditions. Biochemistry. 19(7):1477-84, 1980.
47. Ros S, Santos C R, Moco S, Baenke F, Kelly G, Howell M, Zamboni N, Schulze A. Functional metabolic screen identifies 6-phosphofructo-2-kinase/fructose-2,6-biphosphatase 4 as an important regulator of prostate cancer cell survival. Cancer Discov. 2(4):328-343, 2012.
48. Ros S, Floter J, Kaymak I, Da Costa C, Houddane A, Dubuis S, Griffiths B, Mitter R, Walz S, Blake S, Behrens A, Brindle K M, Zamboni N, Rider M H, Schulze A. 6-Phosphofructo-2-kinase/fructose-2,6-biphosphatase 4 is essential for p53-null cancer cells. Oncogene. 36(23):3287-3299, 2017.
49. Saito T, Rehmsmeier M. The precision-recall plot is more informative than the ROC plot when evaluating binary classifiers on imbalanced datasets. PLoS One 10: e0118432, 2015.
50. Schmitt D L, An S. Spatial organization of metabolic enzyme complexes in cells. Biochemistry. 56(25):3184-3196, 2017.
51. Shorten C, Khoshgoftaar T M. A survey on image data augmentation for deep learning J. Big Data 6:1-48, 2019.
52. Sotgia F, Bonuccelli G, Minetti C, Woodman S E, Capozza F, Kemp R G, Scherer P E, Lisanti M P. Phosphofructokinase muscle-specific isoform requires caveolin-3 expression for plasma membrane recruitment and caveolar targeting: implications for the pathogenesis of caveolin-related muscle diseases. Am J Pathol. 163(6):2619-34, 2003.
53. Tanner L B, Goglia A G, Wei M H, Sehgal T, Parsons L R, Park J O, White E, Toettcher J E, Rabinowitz J D. Four key steps control glycolytic flux in mammalian cells. Cell Syst. 7(1):49-62.e8, 2018.
54. Tatapudy S, Aloisio F, Barber D, Nystul T. Cell fate decisions: emerging roles for metabolic signals and cell morphology. EMBO Rep. 18(12):2105-2118, 2017.
55. Thul P J, Akesson L, Wiking M, Mahdessian D, Geladaki A, Ait Blal H, Alm T, Asplund A, Bjork L, Breckels L M, Backstrom A, Danielsson F, Fagerberg L, Fall J, Gatto L, Gnann C, Hober S, Hjelmare M, Johansson F, Lee S, Lindskog C, Mulder J, Mulvey C M, Nilsson P, Oksvold P, Rockberg J, Schutten R, Schwenk J M, Sivertsson A, Sjostedt E, Skogs M, Stadler C, Sullivan D P, Tegel H, Winsnes C, Zhang C, Zwahlen M, Mardinoglu A, Ponten F, von Feilitzen K, Lilley K S, Uhlen M, Lundberg E. A subcellular map of the human proteome. Science. 356(6340):eaal3321, 2017. (www.proteinatlas.org/ENSG00000141959-PFKL/cell #human and www.proteinatlas.org/ENSG00000114268-PFKFB4/cell)
56 Van Schaftingen E, Jett M F, Hue L, Hers H G. Control of liver 6-phosphofructokinase by fructose 2,6-bisphosphate and other effectors. Proc Natl Acad Sci USA. 1981; 78(6):3483-6.
57. Warburg O, Wind F, Negelein E. The metabolism of tumors in the body. J Gen Physiol. 8:519-530, 1927.
58. Webb B A, Dosey A M, Wittmann T, Kollman J M, Barber D L. The glycolytic enzyme phosphofructokinase-1 assembles into filaments. J Cell Biol. 216(8):2305-2313, 2017.
59. Willars G B, Calthrop-Owen E F, Tomlinson D R. Anterograde and retrograde axonal transport of 6-phosphofructokinase activity in the rat sciatic nerve. Neurochem Int. 10(2):199-203, 1987.
60. Yan W, Wu X, Zhou W, Fong M Y, Cao M, Liu J, Liu X, Chen C H, Fadare O, Pizzo D P, Wu J, Liu L, Liu X, Chin A R, Ren X, Chen Y, Locasale J W, Wang S E. Cancer-cell-secreted exosomal miR-105 promotes tumour growth through the MYC-dependent metabolic reprogramming of stromal cells. Nat Cell Biol. 20(5):597-609, 2018.
61. Yang E, Wang X, Gong Z, Yu M, Wu H, Zhang D. Exosome-mediated metabolic reprogramming: the emerging role in tumor microenvironment remodeling and its influence on cancer progression. Signal Transduct Target Ther. 5(1):242, 2020.
62. Yang J, Li J, Le Y, Zhou C, Zhang S, Gong Z. PFKL/miR-128 axis regulates glycolysis by inhibiting AKT phosphorylation and predicts poor survival in lung cancer. Am J Cancer Res. 6(2):473-85, 2016.
63. Zala D, Hinckelmann M V, Yu H, Lyra da Cunha M M, Liot G, Cordelieres F P, Marco S, Saudou F. Vesicular glycolysis provides on-board energy for fast axonal transport. Cell 152(3):479-491, 2013.
64. Zhang C, Liu J, Wu R, Liang Y, Lin M, Liu J, Chan C S, Hu W, Feng Z. Tumor suppressor p53 negatively regulates glycolysis stimulated by hypoxia through its target RRAD. Oncotarget. 5(14):5535-46, 2014.
65. Zhao F Q. Biology of glucose transport in the mammary gland. J Mammary Gland Biol Neoplasia. 19(1):3-17, 2014.
66. Zhao H, Yang L, Baddour J, Achreja A, Bernard V, Moss T, Marini J C, Tudawe T, Seviour E G, San Lucas F A, Alvarez H, Gupta S, Maiti S N, Cooper L, Peehl D, Ram P T, Maitra 67. A, Nagrath D. Tumor microenvironment derived exosomes pleiotropically modulate cancer cell metabolism. Elife. 5:e10250, 2016.

1. A method comprising:
(a) obtaining a sample from a subject; and
(b) determining intracellular localization of phosphofructokinase type L (PFKL) and/or phosphofructokinase/fructose-2,6-bisphosphatase type 4 (PFKFB4) in cells from the sample.

2. The method of claim 1, further comprising determining intracellular localization of one or more additional biomarkers in the cells of the sample.

3. The method of claim 2, wherein the one or more additional biomarkers includes phosphorylated glucose transporter type 1 (pGLUT1).

4. The method of claim 2, wherein the one or more additional biomarkers includes transketolase-like protein-1, glutathione synthetase, GTP-loaded RhoA, and/or RhoA.

5. The method of claim 1, wherein the subject has cancer or has previously had cancer, and wherein peripheral intracellular localization of PFKL and/or PFKFB4 predicts cancer recurrence.

6. The method of claim 3, wherein the subject has cancer or has previously had cancer, and wherein peripheral intracellular localization of PFKL, PFKFB4, and/or pGLUT1 predicts cancer recurrence.

7. The method of claim 3, wherein the subject has cancer or has previously had cancer, and wherein peripheral intracellular localization of PFKL, PFKFB4, and/or the one or more additional biomarkers predicts cancer recurrence.

8. The method of any of claims 1-7, wherein determining intracellular localization comprises:
a) immunostaining the sample with a primary antibody directed to the biomarker for cancer recurrence; and
b) imaging the sample.

9. The method of claim 8, wherein the primary antibody is detected with a secondary antibody comprising a detectable label.

10. The method of claim 8, wherein imaging the sample comprises fluorescence microscopy.

11. The method of any of claims 1-10, wherein the cancer is selected from breast cancer, prostate cancer, lung cancer, melanoma, kidney cancer, thyroid cancer, pancreatic cancer, stomach cancer or bladder cancer.

12. The method of claim 11, wherein the breast cancer comprises ductal carcinoma in situ of the breast, lobular carcinoma in situ, atypical ductal hyperplasia, or atypical lobular hyperplasia.

13. The method of claim 11 or 12, wherein the cancer recurrence is ipsilateral breast cancer recurrence.

14. The method of any one of claims 1-13, wherein the sample comprises a formalin-fixed paraffin-embedded cancer tissue sample or a cancer metastases tissue or cell sample.

15. The method of any of claims 1-14, further comprising treating a subject predicted to have cancer recurrence with surgery.

16. The method of any of claims 1-14, further comprising treating a subject predicted to have cancer recurrence with administration of inhibitors to enzyme or transporter accumulation at plasma membrane.

17. The method of claim 16, wherein the inhibitors to enzyme accumulation at plasma membrane comprise colchicine, taxol, a calmodulin antagonist, a prenylation inhibitor, an anesthetic, or combinations thereof.

18. A method of preventing cancer recurrence in a subject comprising:
predicting cancer recurrence by the method of one of claims 1-14; and
administering a treatment regimen to prevent cancer recurrence.

19. The method of claim 18, wherein the treatment regimen comprises one or more of: surgery; administration of an inhibitor to enzyme or transporter accumulation at plasma membrane; immunotherapy; radiotherapy; and administration of a chemotherapeutic agent.

20. A method for distinguishing recurrent from non-recurrent cancer comprising determining intracellular localization of a biomarker for cancer recurrence in a sample comprising cancer cells, wherein the biomarker for cancer recurrence comprises phosphofructokinase type L (PFKL) and/or phosphofructokinase/fructose-2,6-bisphosphatase type 4 (PFKFB4), optionally comprises phorsphorylated glucost transporter 1 (pGLUT1), and optionally comprises a biomarker selected from the group consisting of transketolase-like protein-1, glutathione synthetase, GTP-loaded RhoA, and RhoA.

21. The method of claim 20, wherein peripheral intracellular localization of the biomarker for cancer recurrence indicates recurrent cancer.

22. A system comprising at least one or all of:
a primary antibody to a biomarker for cancer recurrence;
an imaging instrument; and
software configured to determine the intracellular location of the biomarker for cancer recurrence; and
optionally, a sample.
